(19) United States
(12) Patent Application Publication
(10) Pub. No.: US 2025/0135166 A1
(43) Pub. Date: May 1, 2025

(54) MULTI-LUMEN BALLOON CATHETER

(71) Applicant: Mauro Carlino, Segrate (IT)

(72) Inventor: Mauro Carlino, Segrate (IT)

(21) Appl. No.: 18/835,781

(22) PCT Filed: Mar. 7, 2023
(86) PCT No.: PCT/EP2023/055752

§ 371 Date: Aug. 5, 2024

(30) Foreign Application Priority Data
Mar. 8, 2022 (IT) 102022000004412

(51) Int. Cl. A61M 25/10; A61M 25/00; A61M 25/09

(52) U.S. Cl. CPC A61M 25/1018; A61M 25/0026; A61M 25/09; A61M 25/104

(57) ABSTRACT

Improved multi-lumen balloon catheter in which the balloon (40) comprises an upper lobe (50) and a lower lobe (52) that extend longitudinally and form a narrowing along the sides, and at least one lumen (46) for the insertion of a secondary guidewire (66) which extends according to a pattern adjacent to the surface (41) of at least one (50) of the lobes to produce fenestrations in the wall of an artery co-localized with the end of the secondary guidewire. The device allows Antegrade Re-entry (TAR) procedures to be performed successfully also by operators who do not have extensive experience in this specific technique.

The present invention relates to a multi-lumen balloon catheter and to an endovascular surgical procedure using said catheter.

### BACKGROUND OF THE INVENTION

The therapeutic object of endovascular intervention is to restore the normal physiological condition of the arteries. For example, due to aging or incorrect diet, the arteries can show a progressive reduction of the lumen useful for passage of the blood flow caused by deposits of fat and plaque.

In the case of stenosis or occlusion of the coronary arteries, the lack of blood flow does not permit the heart to function correctly and can cause irreparable damage.

Coronary disease occurs in two opposite clinical variants. At one end of the spectrum is acute coronary syndrome (ACS), in which narrowing of the lumen of the coronary artery develops rapidly due to thrombotic occlusion that, unless the blood flow is promptly restored, causes myocardial infarction. At the opposite end of the spectrum is chronic angina syndrome (CAS), with a gradual loss of lumen and hence reduction of the blood flow that causes progressive and significant ischemia of the myocardial muscle, with the occurrence of angina and chronic ischemic heart disease.

In some cases, narrowing of the coronary artery can continue until it becomes completely occluded and, through the mechanism of collateral circulation, still not completely understood, can still supply sufficient blood flow to keep the myocardium viable, albeit with a certain degree of ischemia. This is the typical scenario indicated as chronic total occlusion (CTO) of a coronary artery and, when present in an artery that supplies blood to a large portion of the myocardial muscle, a heart surgery can be required to restore the normal blood flow. One of these surgical interventions involves the placement of a coronary stent that expands the artery in the point at which the coronary lesion limits the blood flow with the aim of restoring the patency of the artery and its ability to supply blood to the heart.

While the placement of a coronary stent is relatively simple in the case of chronic angina syndrome (CAS), it is more challenging in the case of chronic total occlusion (CTO) of a coronary artery due to the lack of prompt access to the distal part of the lesion, resulting in the need to use a coronary wire that extends through, or passes around, the plaque of the lesion to act as a guide for the subsequent placement of a stent.

Many procedures are known for treating chronic total occlusion (CTO). In brief, they can be classified as antegrade and retrograde approaches, according to the side of the lesion that is initially attempted to be passed through. The fundamental aim of each approach is to place a guidewire through the lesion, with both ends of the wire arranged inside the lumen of the artery, regardless of the path of the wire inside or around the lesion, so as to allow a stent to be implanted and restore the patency of the artery.

Among the antegrade techniques, Antegrade Dissection and Re-entry (ADR) is currently recommended for occlusions of over 20 mm. Although the adoption of the Stingray™ system (Boston Scientific, Marlborough, MA) has facilitated the execution and degree of success of ADR, the device used requires in-depth and dedicated training of the operators and has a high financial cost. Moreover, its efficacy is further limited by developing a compressive subintimal hematoma.

Carlino et al., Antegrade fenestration and re-entry: A new controlled subintimal technique for chronic total occlusion recanalization; Catheter. Cardiovasc. Interv. 2018; 92:497-504, described for the first time a new ADR technique, called “Antegrade Fenestration and Re-entry” (AFR), which represents an alternative to re-entry based on the Stingray™ system. This technique can be used in three specific clinical scenarios: i) as first-line ADR; ii) as an alternative to an antegrade approach when however, the wire is advanced subintimal; iii) as alternative in other ADR techniques, such as in the case of failed Crossboss/Stingray system. The AFR technique has five steps, as described in the aforesaid article by Dr Carlino and collaborators:
Step 1: The guidewire inadvertently follows a subintimal course around the occlusion and is located in the subintimal space beyond the distal portion of the occlusion.
Step 2: the microcatheter is removed (with conventional techniques), leaving the first guidewire in place, and a second guidewire is inserted, again through the subintimal space around the occlusion, keeping the tip of the second guidewire as close as possible to the first wire, checking in several projections.
Step 3: a balloon size 1:1 with the artery diameter is advanced on the first guidewire and is placed through the distal portion of the occlusion.
Step 4: the balloon is inflated to at least nominal pressure.
Step 5: the balloon is deflated, and the second guidewire is advanced rapidly through the temporary openings between the subintimal space and the true lumen, called “fenestrations”, created by the balloon before its flaps, and hence the fenestrations are closed again in this way allowing the insertion of the secondary guidewire into the true lumen of the artery, which is now accessible.

US 2014/0277053 A1 discloses a subintimal re-entry catheter with shape-controlled balloon. The catheter includes an elongated shaft and an inflatable balloon mounted on a distal region of the elongated shaft. The catheter comprises a guidewire lumen, and an inflation lumen and may include a re-entry device lumen in communication with a lateral port of the catheter. The inflation lumen is in communication with the inflatable balloon, configured to orient the lateral port of the catheter toward the lumen of the blood vessel. The balloon is also configured to be inflated to a first and a second inflation state. A guidewire or an elongated penetration member is advanced through the guidewire lumen and the lateral port of the catheter until it penetrates into the true lumen distal of an occlusion so that a therapeutic procedure is performed. The body of the balloon does not comprise any lumen for any wire or penetration member.

US 2017/0100141 A1 discloses an occlusion bypassing apparatus for re-entering the true lumen of a vessel. The apparatus comprises an outer shaft with a needle lumen, a side port at a distal end, a needle and an inflatable balloon. A curved portion of the needle housing allows the needle to exit the side port with a correct orientation for re-entry of a true lumen of a vessel. The body of the balloon does not comprise any lumen for any wire or penetration member.

A conventional balloon catheter, according to the prior art, is shown in FIG. 1 and designated with 20.

WO 2019/112781 A1 describes a device and a method for passing beyond a chronic total occlusion (CTO) in an artery with an AFR technique as described above. FIGS. 2-4 correspond respectively to FIGS. 3, 5 and 6 of WO 2019/112781 A1 and show the steps of the AFR technique implemented with the balloon catheter described in this document, which is set forth briefly below.

FIG. 2 shows the catheter 20 with balloon 26, a primary guidewire 22 inserted into the main lumen of the catheter and protruding from the distal end thereof, and a secondary guidewire 24 inserted into a secondary lumen of the catheter and protruding from a hole upstream of the balloon. The balloon catheter and the primary and secondary guidewires have been inserted into the true lumen 12 of an artery 14, then advanced through the subintimal space 16 of the artery so that the balloon is placed at the chronic total occlusion CTO 18. This placement is obtained with Steps 1, 2 and 3 of the AFR technique mentioned above.

FIG. 3 shows balloon inflation, as in Step 4 of the AFR technique.

FIG. 4 shows balloon inflation 26, obtained with means and devices not shown as they are known to the person skilled in the art. The consequent formation of at least one fenestration 29, the flaps 28, 28′ of which are provisionally open allowing the secondary guidewire 24 to be advanced and re-enter the true lumen 12, as in Step 5 of the AFR technique. A stent, which will push the plaque outward, restoring the patency of the vessel, as shown in FIG. 10, is then advanced on the secondary guidewire.

The AFR technique described above has some important and specific features.

Firstly, while the operator can select the primary guidewire according to his/her preferences, the secondary guidewire must be a guidewire with a polymer coating and with a low tip load, as it must be maneuvered rapidly to pass easily through the fenestrations 29 created between the false lumen of the subintimal space 16 and the true lumen 12, avoiding the risk of perforating the vessel.

Secondly, re-entry into the true lumen 12 is an iterative process, and it may take some attempts before managing to pass through a fenestration effectively.

Thirdly, re-entry into the true lumen 12 must be performed as close as possible to the distal portion of the occlusion and far from the attachment of the side branches of the vessels. This allows minimization of the subintimal path and of the risk of losing the side branches, and maximization of the likelihood of obtaining a good distal runoff, all key factors in the success of Percutaneous Coronary Intervention (PCI) for the treatment of CTO based on AFR, and ensures long term patency rates. Fourthly, the secondary guidewire must initially be advanced as close as possible to the first guidewire in order to increase the likelihood of passing through a fenestration created by balloon inflation.

Therefore, execution of AFR requires considerable skill and expertise of the operator, and in fact the technique only had a 66% success rate in a multicenter validation study described by Azzalini et al., Multicenter experience with the antegrade fenestration and reentry technique for chronic total occlusion recanalization; Catheter. Cardiovasc. Interv. 2021;97: E40-E50. U.S. Pat. No. 5,342,301 describes balloon catheters in which the balloon is of the multi-lumen type to allow the passage of guidewires, glass fiber bundles, or other devices.

Therefore, there is a need for a new device and technique that capitalize on the lesson learned from AFR but improve the AFR success rates by substantially eliminating those steps in AFR that have been associated with failure to re-cross from the subintimal space into the true lumen, ultimately, increase the likelihood of success of recanalization by operators with less expertise.

### SUMMARY OF THE INVENTION

An object of the present invention is to provide an improved multi-lumen balloon catheter that allows a new and different iteration of what the author inventor has previously called Antegrade Fenestration and Re-entry (AFR) procedures. This new and never described device and technique are intended to be successfully used by operators who do not have in-depth expertise in complex coronary intervention. The particular nature of this new device eliminates a key step of the AFR, namely the necessity to create “a fenestration” with its complex tridimensional nature and its transient and variable ability to connect subintimal space with true lumen only for a limited amount of time. This obligates the crossing wire to engage them within a limited time (30 sec based on the relevant literature) in order to cross from the subintimal space into the true lumen. The improvement associated to the present invention has been achieved thanks to the co-localization of the secondary wire (tasked to cross from subintimal into true lumen) on the balloon responsible for the mechanical disruption of the membrane that separates subintimal space from true lumen.

To further effectively innovate the known device and set it apart from any prior device and technique, the wire aiming to re-enter into the true lumen is itself the agent that causes a targeted perforation of the subintimal membrane resulting from the limited and operator-directed wire exposure out of the balloon, on the balloon surface and prior to balloon inflation. The secondary wire (i.e., crossing wire) is propelled against the sub-intimal membrane by the combined action of the balloon inflation, the particular shape (nose shape or linear lumen on the superior aspect of the balloon) of the underlying balloon at the point where the wire exits the balloon. The presence of a wire in contact and on the surface of the expanding balloon changes the expansion profile and compliance of the balloon locally to further enhance the selective disruptive action of the balloon just in the presence of the wire and therefore biasing the wire into re-entry across the membrane in the true lumen.

Prior AFR techniques and devices that attempted to fenestrate the subintimal membrane did not take advantage of the crossing wire as an agent to target and cause selective targeted perforation of the subintimal membrane into the membrane in the location chosen by the operator and the immediate presence of the crossing wire. AFR, as previously described, left to the extensive subintimal disruption and wire-operator ability to journey this subintimal maze the chance to find a way into the true lumen eventually (wire journey).

The aforementioned wire journey takes a finite amount of time for the tip of the wire to leave the device shaft, run over the deflated balloon, engaging and (hopefully) find a patent fenestration to re-engage into the true lumen as described in known devices.

In the device presented in this application and its relative use (or technique), the amount of time required to perform the “wire journey is zero, i.e., none of the steps described in the wire journey exists nor are required any longer since the wire that causes the localized perforation of the subintimal membrane is already ahead of the balloon when it crosses into the true lumen. In Target Antegrade Re-Entry (TAR) the wire is delivered directly to the tip (or other exit points). The wire is exposed in the subintimal space (like in many other CTO techniques, e.g., re-CART, parallel wiring, LAST), the balloon is inflated, and as a result of its own action, the wire is propelled by the balloon inflation against and through the membrane into the true lumen. Through the profound conceptual transformation of AFR into TAR, the only common feature that TAR and AFR share is the use of a dual lumen catheter and the presence of a balloon in subintimal space, as many other devices and techniques do.

Another object of the invention is to provide an endovascular surgical procedure of a new re-entry technique (TAR) that, thanks to the improved multi-lumen balloon catheter, is more effective and simpler than AFR as previously described.

Therefore, an aspect of the present invention relates to a multi-lumen balloon catheter comprising:
a catheter provided with a first lumen for the insertion of a primary guidewire, a second lumen for the insertion of a secondary guidewire, and a third lumen for balloon inflation;
a multi-lumen inflatable balloon comprising a first lumen that extends through the balloon from the proximal end to the distal end and is in communication with said first lumen of said catheter, and at least a second lumen, for insertion of said secondary guidewire;
   characterized in that:
said balloon comprises an upper lobe and a lower lobe located respectively above and below a horizontal plan that intersects the balloon in the portion between said lobes, said lobes extending longitudinally and forming a narrowing along the sides of the balloon,
said second lumen of said balloon extends from said proximal end to said distal end according to a pattern adjacent and parallel to the surface of at least one of said upper or lower lobes, said second lumen of said balloon being in communication with said second lumen of said catheter,
said second lumen of said catheter is in communication with a pair of lateral exit holes upstream of the proximal end of said balloon.

According to an aspect of the invention, the second lumen of the balloon is in communication with at least one exit hole on said surface of one of the upper or lower lobes and is arranged upstream of the distal end of the balloon. In particular, the exit hole is provided in the distal half of the balloon with respect to a centreline ideally dividing the balloon into a proximal half and a distal half.

According to an aspect of the invention, the balloon comprises a third lumen extending from the proximal end to the distal end according to a pattern adjacent to the surface of the other of said upper or lower lobe, and originates from a bifurcation which is in communication with said second lumen of said catheter.

According to an aspect of the invention, the third lumen of said balloon is in communication with at least one exit hole on the surface of the other of said upper or lower lobe, and is arranged upstream of the distal end of the balloon. In particular, the exit hole is provided in the distal half of the balloon with respect to a centreline, ideally dividing the balloon into a proximal half and a distal half.

Another aspect of the invention concerns an endovascular surgical procedure to place a guidewire downstream of a total occlusion in a patient's artery, comprising:
advancing into the subintimal space of said artery a primary guidewire and placing a multi-lumen inflatable balloon catheter at said total occlusion, wherein said balloon comprises a first lumen that hosts said primary wire and at least a second lumen for hosting a secondary guidewire;
advancing said secondary guidewire into said second lumen of said balloon and exposing its tip outside said balloon to create targeted perforation of the subintimal membrane into the true lumen of said artery or to pierce the subintimal space and intimal layer to penetrate into the true lumen;
inflating and deflating the balloon to exert selective stresses on said subintimal space and create at least one targeted perforation of the subintimal membrane;
introducing said secondary guidewire into said targeted perforation of the subintimal membrane
   and advancing it within the true lumen downstream of said total occlusion.

### BRIEF DESCRIPTION OF THE DRAWINGS

FIGS. 1-4 show balloon catheters according to the prior art in various operating arrangements;

FIG. 5 is a side elevation view of a balloon catheter according to a first embodiment of the invention;

FIG. 6 is a perspective view of the balloon catheter of FIG. 5;

FIG. 7 is a sectional view along the line VII-VII of FIG. 6;

FIG. 8 is a partial sectional view of a balloon catheter according to a second embodiment of the invention;

FIG. 9 is a longitudinal sectional view of a balloon catheter according to a third embodiment of the invention; and

FIG. 10 is a longitudinal sectional view of a stent applied to a blood vessel.

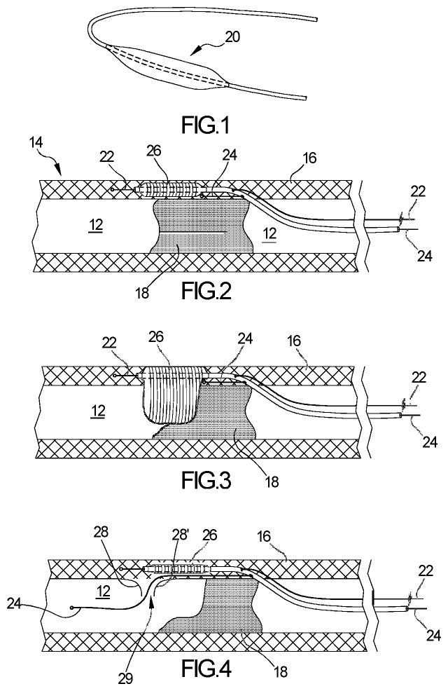

### DETAILED DESCRIPTION OF THE INVENTION

The present invention relates to a multi-lumen balloon catheter for performing Target Antegrade and Re-entry (TAR) procedures in blood vessels, in particular in the coronary arteries.

The Antegrade Fenestration and Re-entry (AFR) procedure in blood vessels, in particular in the coronary arteries, has been described briefly in the introductory part of the present description relating to state of the art. This procedure (Target Antegrade Re-entry, TAR) shall be referred to again in the description relating to using the new multi-lumen balloon catheter according to the present invention.

In the description of the balloon catheter and of the TAR procedure using said balloon catheter the following terms have the meaning set forth below:
“balloon catheter” designates a catheter provided with a balloon that can be inflated and deflated several times;
“proximal end” indicates the end of the balloon closer to the operator and “distal end” indicates the end of the balloon farther from the operator;
“longitudinal extension” of the balloon indicates the extension from the proximal end to the distal end;
“lumen” indicates both the channels present in the catheter and in the balloon and the cavities of the blood vessels;
“true lumen” indicates the natural cavity of the blood vessel and “false lumen” indicates the cavity created in the subintimal space by the advancement of the balloon catheter;
the terms “primary guidewire” and “primary wire” are used interchangeably and have the same meaning. This is also the case for the terms “secondary guidewire” and “secondary wire”;
upper lobe of the balloon is the lobe located above a horizontal plane A-A that intersects the balloon in the portion between said lobes (FIG. 7);
lower lobe of the balloon is the lobe located below a horizontal plane A-A that intersects the balloon in the portion between said lobes (FIG. 7).

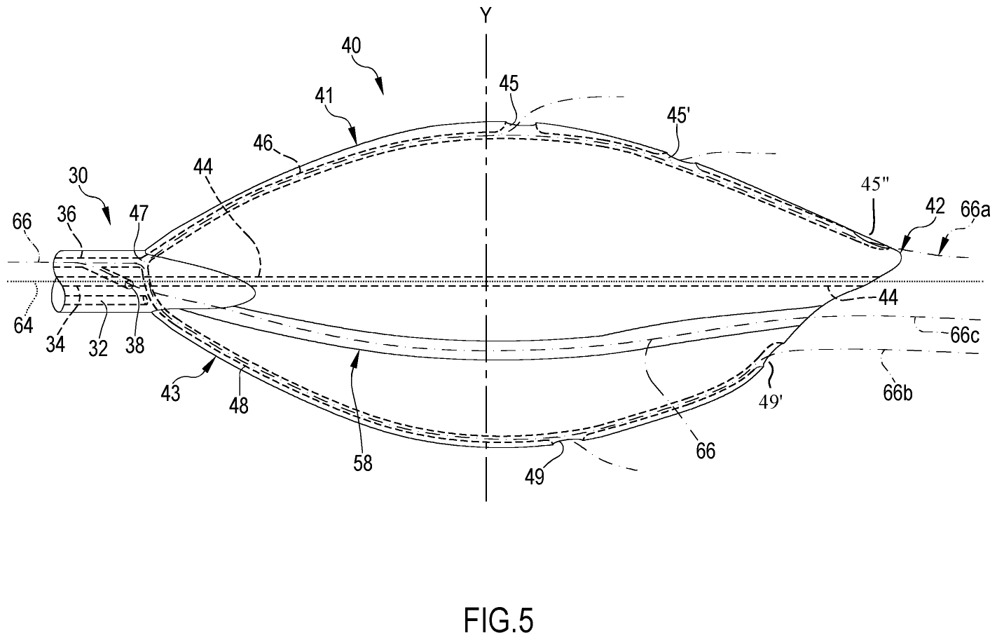

With reference to FIGS. 5-7, the balloon catheter, according to the invention, comprises a microcatheter 30, of which only the terminal part connected to the proximal end of the balloon is shown, and a balloon 40 of elongated shape, tapered at the proximal and distal ends, this latter being formed with a nose 42.

Balloon 40 is of the multi-lumen type. It comprises a first lumen 44 that extends through the balloon along its longitudinal axis from the proximal end to the distal end, and a second lumen 46 which also extends from the proximal end to the distal end of the balloon but according to a pattern adjacent to the upper surface 41 of the balloon. The term “pattern adjacent to the upper surface 41 of the balloon” means that when the balloon is in an inflated state the second lumen 46 runs substantially parallel to the upper surface 41 of the balloon, and in proximity thereof, i.e., the distance between a point of the surface and a point of the second lumen with common normal is substantially constant.

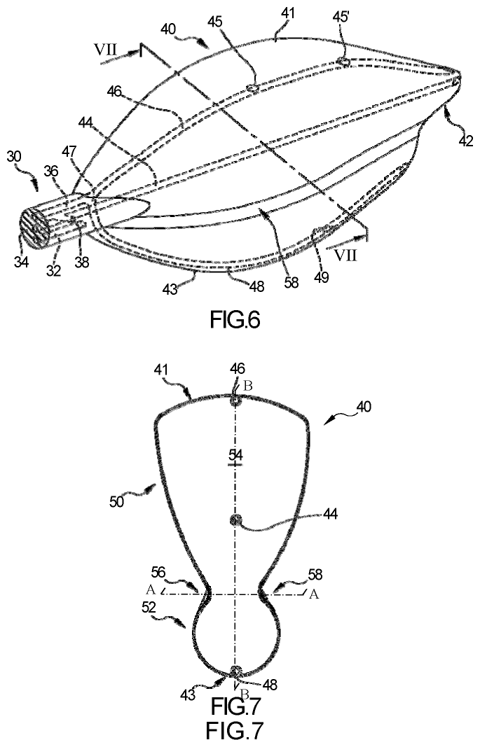

With reference in particular to FIGS. 5 and 6, the nose 42 of the balloon is the distal tip of the balloon and is formed above the first lumen 44.

As shown in FIG. 5, the second lumen 46 emerges on the nose 42 in a point farther forward with respect to the point in which the first lumen 44 emerges, i.e., in a more distal position with respect to the point in which the first lumen 44 emerges.

In the embodiment illustrated, a third lumen 48 is also present, which also extends from the proximal end to the distal end of the balloon but according to a pattern adjacent to the lower surface 43 of the balloon.

The term “pattern adjacent to the lower surface 43 of the balloon” means that when the balloon is in an inflated state the third lumen 48 runs substantially parallel to the lower surface 43 of the balloon, and in proximity thereof; i.e., the distance between a point of the surface and a point of the third lumen with common normal is substantially constant.

The terms “upper surface” and “lower surface” of the balloon are defined below.

The second lumen 46 and the third lumen 48 have a common origin in the bifurcation 47 that connects the two lumens 46, 48 of the balloon to the lumen 36 of the catheter 30. The second lumen 46 is provided with holes 45, 45′ 45″ and the third lumen 48 is provided with at least one hole 49, respectively on the upper surface 41 and on the lower surface 43 of the balloon. With reference to FIG. 5, a centreline Y-Y ideally divides the balloon into a proximal half, connected to the catheter 30, and a distal half, terminating in the nose 42. The holes 45, 45′45″ and 49 are provided in the distal half of the balloon 40 with respect to the centreline Y-Y.

The second lumen 46 is also provided with a hole 45″ at the distal end of the balloon, above nose 42, and the third lumen 48 is also provided with a hole 49′ at the distal end of the balloon, below nose 42.

With reference in particular to FIG. 7, the balloon 40 is formed with an upper lobe 50 and a lower lobe 52, that extend longitudinally for the whole of the extension of the balloon, from the proximal end to the distal end. A narrowing, which forms two longitudinal channels on the sides of the balloon, is defined in the portion between the two lobes 50, 52. With reference to FIG. 7, which is a view towards the distal end of the balloon, the channels are designated as left channel 56 and right channel 58.

For a better definition of the shape and parts of the balloon 40, line A-A defines a horizontal plane that intersects transversally the balloon in the portion between the two lobes 50, 52, and line B-B defines a vertical plane that intersects longitudinally the balloon 40 through its entire length. Therefore, lobe 50 is defined as the upper lobe and lobe 52 is defined as the lower lobe, irrespectively of the actual position and orientation that the lobes will have when the balloon is inflated in an artery.

The term “upper surface” 41 of the balloon designates the upper surface of the upper lobe 50 of the balloon 40, while the term “lower surface” 43 of the balloon designates the lower surface of the lower lobe 52 of the balloon 40.

With reference to the vertical plane B-B, when looking the balloon from a proximal end, i.e., from the end of the balloon closer to the operator, plane B-B divides the balloon on a left half and a right half.

According to an embodiment, the lobes 50, 52 are not the same size. In the embodiment illustrated, the upper lobe 50 has a larger volume than that of the lower lobe 52. Moreover, the first lumen 44 is contained in the lobe of larger size 50. Although FIG. 7 shows that the lumens 44, 46 and 48 are aligned on the same vertical axis, in other embodiments, not illustrated, the second lumen 46 and the third lumen 48 may not be aligned with the lumen 44, albeit always adjacent to the upper and lower surface, respectively.

The balloon 40 is inflatable by the introduction of a gas or of a suitable liquid into the cavity 54, as is known in the art.

The catheter 30 is provided with at least three lumens. A first lumen 34 aligned with the first lumen 44 of the balloon, for the insertion of a primary guidewire 64, a second lumen 36 for insertion of at least one secondary guidewire 66, and a third lumen for balloon inflation, designated with 32. In an embodiment, the catheter is a tube with a diameter ranging from 1.8-3.1 Fr, where 1 Fr=0.33 mm. The primary guidewire is for the delivery of the device at the chronic total occlusion CTO, while the secondary wire is for the insertion into the true lumen of the artery.

The second lumen 36 is also provided with a pair of exit holes 38 upstream of the proximal end of the balloon, placed laterally on the catheter. FIGS. 5 and 6 show only one of the lateral holes 38, but a corresponding hole is provided in a diametrically opposite position. The lateral positioning of the holes 38 is referred to the sides of the balloon, i.e., both these holes are substantially aligned with the longitudinal channels 56, 58 on the sides of the balloon 40.

The secondary guidewire 66, after being introduced into the lumen 36 of the catheter, can be made to protrude from one of the exit holes 38 upstream of the proximal end of the balloon, or can be advanced to the bifurcation 47, and from here can be introduced by the operator either into the second lumen 46 or into the third lumen 48 of the balloon, as will be described below. If the secondary guidewire 66 is made to protrude from one of the lateral holes 38 of the catheter upstream of the balloon, it can be advanced inside and along the longitudinal channels 56, 58 on the outer sides of the balloon 40, as will be explained below.

FIG. 8 shows a second embodiment of the balloon catheter according to the invention, in which the distal end is rounded in shape, and there is no protruding nose as in the embodiment described previously.

Maintaining the same reference numbers used in relation to the embodiment described previously, the second lumen 46 and the third lumen 48 are present in the balloon 40, as is the first lumen for insertion of the primary guidewire, not illustrated.

In this embodiment, the second lumen 46 protrudes from the interior of the balloon in the distal half and is contained inside an outer rib 47 which protrudes from the upper surface 41 of said balloon. In a similar variant, not illustrated, the outer rib is formed symmetrically on the lower surface 43 of the balloon, defining an analogous pattern of the third lumen 48.

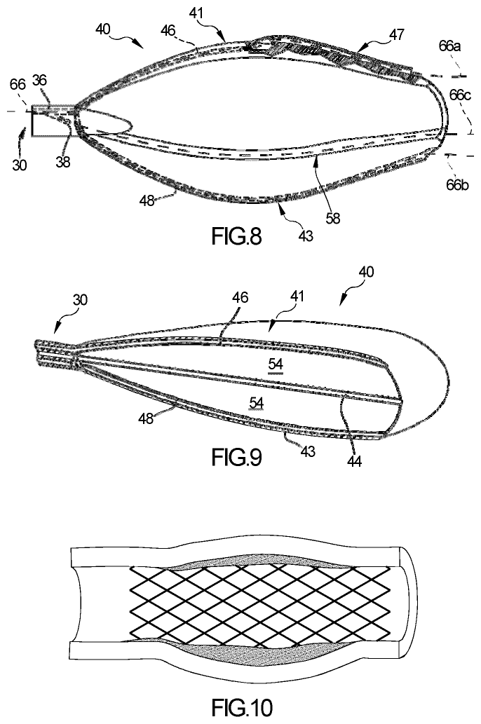

FIG. 9 shows a sectional view of a third embodiment of the balloon catheter according to the invention, in which the distal end is once again rounded in shape and not provided with a nose and protruding rib. The first lumen 44, the second lumen 46 and the third lumen 48 of the balloon, as well as the inner cavity 54, are shown.

The balloon catheter is used to perform an endovascular surgical procedure of TAR, according to a method that forms another aspect of the invention. The object of the procedure is to create one or more connections between the subintimal space and the true lumen by performing targeted perforation of the subintimal membrane, suitable to allow introduction of the secondary guidewire into the true lumen of the artery.

The balloon of the invention is used to create these connections both in non-specific points and in specific points of the region around the balloon, with various mechanisms.

With reference to the various steps of the TAR technique mentioned in the introductory part of the description, and to FIGS. 5-8, it must be considered that a primary guidewire 64, introduced into the first lumen 34 of the catheter 30 and into the primary lumen 44 of the balloon 40, has been advanced in a subintimal path around the total occlusion (CTO) 18 of FIG. 2, and is located in the subintimal space beyond the distal portion of the occlusion, as shown, for example, in FIG. 2 for a conventional catheter.

With reference in particular to FIGS. 5 and 8, a secondary wire 66, introduced into the second lumen 36 of the catheter, is advanced to the bifurcation 47 to then engage the second lumen 46 of the balloon and protrude from it on the nose 42, with its end, or tip, above the primary guidewire 44. In this case, the tip of the secondary wire 66 is designated as 66a. This path of the secondary wire is defined “upper”.

Alternatively, upon reaching the bifurcation 47, the operator can choose to advance the secondary guidewire 66 into the third lumen 48 of the balloon, and make it protrude from the distal end of the balloon with its tip below the primary guidewire 44. In this case, the tip of the secondary wire 66 is designated as 66b. This path of the secondary wire is defined “lower”.

The structure of the catheter and of the balloon of the invention provide the operator with another opportunity for advancement of the secondary wire 66, represented by the exit from the catheter 30 through one of the lateral holes 38 upstream of the balloon 40. In this case, the secondary guidewire 66 is then advanced outside the balloon along one of the channels 56, 58 defined by the narrowing of the two lobes 50, 52, until reaching the distal end of the balloon. In this case, the tip of the secondary wire 66 is designated as 66c. This path of the secondary wire is defined “lateral”.

The balloon catheter of the invention allows perforation to be created in specific regions, in which also the tip of the secondary guidewire 66 is placed and exposed, so that it can be immediately introduced into a opening of the subintimal membrane and hence into the true lumen 12, as explained below.

### Technique 1 (“Cut Through”)

A longitudinal linear fenestration is created along the longitudinal axis of the balloon from the upper path of the secondary wire 66 into the second lumen 46 of the balloon, or from the lower path of the secondary wire 66 into the third lumen 48 of the balloon, as these areas of the balloon exert a greater pressure on the intima layer during inflation. This pressure is further increased in the case in which the first or second lumen are inside a rib protruding from the surface of the balloon, as shown in FIG. 8.

Moreover, the holes 45, 45′ or 49, respectively on the upper surface 41 and on the lower surface 43 of the balloon, allow the tip of the secondary guidewire to exit the balloon and be exposed in various points on the surface of the balloon, selected by the operator, so as to obtain close proximity with the point in which the targeted perforation of the subintimal membrane has been created. This allows “co-localization of the tip of the secondary wire and of the perforation”, which is a fundamental feature of the device and of the method according to the invention. In this way, the temporary nature of the fenestrations, previously described as part of the key feature of the AFR technique, is no longer a limiting factor, as the wire is co-localized with the connection when these are formed, and its immediate vicinity favors insertion into the true lumen.

### Technique 2 (“Punch Through”)

A targeted perforation of the subintimal membrane to establish a direct connection between subintimal space and true lumen is created from a break point distal from the secondary wire 66 that protrudes from the terminal hole of the second lumen 46 of the balloon, beyond the nose 42, with tip of the wire designated as 66a and leaving exiting from the balloon from hole 45″. Inflation of the balloon propels the conical nose 42 and the tip 66a of the secondary wire against and beyond the membrane of the cul-de-sac, opening a perforation and causing the wire 66, which is co-localized with the perforation to pass through it, carrying the wire into the true lumen. Also in this case the principle of co-localization of the perforation and the tip of the wire is met.

### Technique 3 (“Horizontal Slice Through”)

A frontal linear perforation on the membrane interposed between subintimal space and true lumen is created along the cross section of the balloon on the intima membrane-cul-de-sac. The secondary wire 66 protrudes from the terminal hole 45″ of the second lumen 46 of the balloon, beyond the nose 42, with tip of the wire designated with 66a having a variable rigidity selected by the operator. In this case, a cycle of balloon inflation/deflation determines a vertical movement of the tip of the secondary wire, i.e., perpendicular to the longitudinal extension of the balloon, which “scratches” the distal membrane of the cul-de-sac exerting a repeated serial vertical pressure that ultimately causes a tear, hence a linear perforation. The tip 66a is in position to pass through the opening and enter the true lumen. Here too the principle of co-localization of the perforation and the secondary wire is met.

### Technique 4 (“Side Slide”)

Lateral engagement: this is applied when the upper or lower path of the secondary wire has not created a connection between the subintimal space and true lumen that can be passed through. In this case the secondary guidewire is retracted into the catheter 30, readvanced and made to exit from one of the lateral holes 38 of the catheter upstream of the balloon. The channels 56, 58 on the outer sides of the balloon, which are preferably coated with a hydrophilic material, guide the sliding movement of the secondary wire on the side of the balloon and help the operator to place the wire 66 along one side of the balloon. The balloon is left inflated to stretch and stabilize perforations into the subintimal membrane obtained by the previously combined action of balloon inflation while the wire was exposed. In any point, the secondary wire 66, with tip 66c, can be advanced beyond the balloon to engage and pass through the openings on the right or left sides, not accessible from the upper or lower surface for anatomical reasons. The principle of co-localization of the perforations and passing through of the wire is also applied in this technique. To be noted that this step is likely the last maneuver to be attempted if other techniques be not successful. A significant difference from AFR, as previously described is that this technique requires the balloon to remain inflated to allow the wire to enter across the subintimal space.

Each technique from 1 to 4 allows the creation of a targeted perforation of the subintimal membrane to establish a direct connection between the subintimal space and the true lumen in predictable areas along the profile of the balloon. This specific feature is peculiar to the device of the invention, allowing the operator to concentrate his/her efforts on inserting the secondary wire in specific regions of the balloon, unlike prior art devices, which probe the cul-de-sac randomly with a wire after multiple inflations of the balloon. The fundamental feature is co-localizing the tip of the secondary wire and the target re-entry zone, and ability of this device to eliminate the entire wire-journey steps (see above) as described in the original AFR technique. As mentioned above, instead of creating transient fenestrations as described in the original AFR (Azzalini et al, 2018), each technique from 1 to 4 can simply pierce the subintimal space and intimal layer and let the secondary wire penetrate into the true lumen.

The advancement techniques #1 #2 and #3 should be performed with the balloon deflated or inflated (i.e. wheter the balloon is inflated—or not—is not a relevant aspect of the technique any longer)

The advancement technique #4 must be performed with the balloon inflated.

When the secondary wire 66 has re-entered the true lumen 12, a stent is then advanced thereon, which will push the plaque outward, restoring the patency of the vessel, as shown in FIG. 10. The use of the multi-lumen balloon catheter according to the invention is not limited to surgical interventions in coronary vessels. In fact, the device can be used in surgical interventions of vascular nature in any region of the artery and/or vein, for recanalization of complete occlusion of various types, calcified or thrombotic, or of a perforation, in peripheral vessels, e.g., iliac, infra-and/or supra-popliteal, subclavian.

More generally, the device can be used in any area of the body in the presence of a complete occlusion or of a perforation of various types, both acute and chronic, of a tubular structure, such as a common bile duct, which requires a recanalization procedure.

In fact, the multi-lumen balloon catheter according to the invention has the specific ability to: 1) create a limited dissection in the vessel wall; 2) create and/or maintain, along the two lateral grooves, with the balloon inflated, an access to the lumen beyond the occlusion by a guidewire/catheter, e.g., ERCP/occlusion of the biliary tract.

1-13. (canceled)

14. A multi-lumen balloon catheter comprising:
a catheter provided with a first lumen for insertion of a primary guidewire, a second lumen for insertion of a secondary guidewire, and a third lumen for balloon inflation;
an inflatable multi-lumen balloon comprising a first lumen that extends though the balloon from a proximal end to a distal end and is in communication with the first lumen of the catheter, and at least one second lumen, for insertion of the secondary guidewire;
wherein the balloon comprises an upper lobe and a lower lobe located respectively above and below a horizontal plane that intersects the balloon in a portion between the upper and lower lobes, the upper and lower lobes extending longitudinally and forming a narrowing along sides of the balloon;
wherein the second lumen of the balloon extends from the proximal end to the distal end according to a pattern adjacent and parallel to a surface of at least one of the upper and lower lobes;
wherein the second lumen of the balloon is in communication with the second lumen of the catheter; and
wherein the second lumen of the catheter is in communication with a pair of lateral exit holes upstream of the proximal end of the balloon.

15. The multi-lumen balloon catheter of claim 14, wherein the second lumen of the balloon is in communication with at least one exit hole on the surface of at least one of the upper and lower lobes of the balloon, and wherein the at least one exit hole is provided in a distal portion of the balloon with respect to a centreline dividing the balloon into a proximal portion comprising the proximal end and a distal portion comprising the distal end.

16. The multi-lumen balloon catheter of claim 14, wherein the balloon comprises a third lumen extending from the proximal end to the distal end of the balloon according to a pattern adjacent to a lower surface of the lower lobe, wherein the second and third lumens of the balloon originate from a bifurcation which is in communication with the second lumen of the catheter.

17. The multi-lumen balloon catheter of claim 14, wherein the balloon is tapered at the proximal and distal ends, the distal end being formed with a nose which protrudes above the first lumen.

18. The multi-lumen balloon catheter of claim 14, wherein the second lumen of the balloon protrudes from an interior of the balloon in a distal portion comprising the distal end, forming an outer rib which protrudes from an upper surface of the balloon.

19. The multi-lumen balloon catheter of claim 14, wherein the third lumen protrudes from an interior of the balloon in a distal portion comprising the distal end, forming an outer rib protruding from a lower surface of the balloon.

20. The multi-lumen balloon catheter of claim 14, wherein the second lumen of the balloon is in communication with at least one exit hole on an upper surface of the upper lobe of the balloon, wherein the at least one exit hole on the upper surface is provided in a distal portion comprising the distal end of the balloon with respect to a centreline that ideally divides the balloon into the distal portion and a proximal portion comprising the proximal end.

21. The multi-lumen balloon catheter of claim 14, wherein the third lumen is in communication with at least one exit hole on a lower surface of the lower lobe of the balloon, wherein the at least one exit hole on the lower surface is provided in a distal portion comprising the distal end of the balloon with respect to a centreline that ideally divides the balloon into the distal portion and a proximal portion comprising the proximal end.

22. The multi-lumen balloon catheter of claim 14, wherein the upper lobe has a volume greater than a volume of the lower lobe.

23. An endovascular surgical method of Target Antegrade and Re-entry (TAR) to place a guidewire downstream of a total occlusion in an artery of a patient, the method comprising:
advancing a primary guidewire into a subintimal space of the artery and placing a multi-lumen inflatable balloon catheter at the total occlusion, wherein the balloon comprises a first lumen that hosts the primary guidewire and at least a second lumen for hosting a secondary guidewire, the balloon being shaped with a nose protruding above the first lumen;
advancing a secondary guidewire into the second lumen of the balloon and exposing its tip outside the balloon at parts of the subintimal space suitable to create targeted perforation of the subintimal membrane to establish a direct connection between the subintimal space and a true lumen of the artery or to pierce the subintimal space and intimal layer to penetrate into the true lumen;
inflating and deflating the balloon to exert selective stresses on the subintimal space and create at least one targeted perforation of the subintimal membrane to establish a direct connection between the subintimal space and true lumen;
introducing the secondary guidewire into the targeted perforation of the subintimal membrane to establish a direct connection between subintimal space and true lumen and advancing it within the true lumen immediately downstream of the total occlusion.

24. The endovascular surgical method of claim 23, wherein the secondary wire is advanced into the second lumen and its tip is exposed prior to balloon inflation from the nose of the balloon to be propelled against and across the membrane upon inflation to create targeted perforation of the subintimal membrane to establish a direct connection between the subintimal space and true lumen

25. The endovascular surgical method of claim 23, wherein the secondary wire is advanced into the second lumen and its tip is exposed from holes of the upper surface or the lower surface of the balloon.

26. The endovascular surgical procedure method of claim 23, wherein the secondary wire is exposed from a lateral hole of the catheter provided upstream of the balloon, and is advanced along a longitudinal channel on the outer side of the balloon while the balloon is inflated to engage targeted perforation of the subintimal membrane previously created but failed to be engaged to re-entry into the true lumen.

27. The endovascular surgical procedure method of claim 23, wherein:
the balloon comprises an upper lobe and a lower lobe located respectively above and below a horizontal plane that intersects the balloon in a portion between the upper and lower lobes, the upper and lower lobes extending longitudinally and forming a narrowing along sides of the balloon; and
the second lumen of the balloon extends from the proximal end to the distal end according to a pattern adjacent and parallel to a surface of at least one of the upper and lower lobes.

28. The endovascular surgical procedure method of claim 27, wherein the second lumen of the balloon is in communication with at least one exit hole on the surface of at least one of the upper and lower lobes of the balloon, and wherein the at least one exit hole is provided in a distal portion of the balloon with respect to a centreline dividing the balloon into a proximal portion comprising the proximal end and a distal portion comprising the distal end.

29. The endovascular surgical procedure method of claim 27, wherein the balloon comprises a third lumen extending from the proximal end to the distal end of the balloon according to a pattern adjacent to a lower surface of the lower lobe, wherein the second and third lumens of the balloon originate from a bifurcation which is in communication with the second lumen of the catheter.

30. The endovascular surgical procedure method of claim 27, wherein the balloon is tapered at the proximal and distal ends, the distal end being formed with a nose which protrudes above the first lumen.

31. The endovascular surgical procedure method of claim 27, wherein the second lumen of the balloon protrudes from an interior of the balloon in a distal portion comprising the distal end, forming an outer rib which protrudes from an upper surface of the balloon.

32. The endovascular surgical procedure method of claim 27, wherein the third lumen protrudes from an interior of the balloon in a distal portion comprising the distal end, forming an outer rib protruding from a lower surface of the balloon.

33. The endovascular surgical procedure method of claim 27, wherein the second lumen of the balloon is in communication with at least one exit hole on an upper surface of the upper lobe of the balloon, wherein the at least one exit hole on the upper surface is provided in a distal portion comprising the distal end of the balloon with respect to a centreline that ideally divides the balloon into the distal portion and a proximal portion comprising the proximal end.
